(19) United States
(12) Patent Application Publication
(10) Pub. No.: US 2017/0011177 A1
(43) Pub. Date: Jan. 12, 2017

(54) AUTOMATED HEALTHCARE INTEGRATION SYSTEM

(71) Applicant: TELECARE GLOBAL, LLC, Leavenworth, KS (US)

(72) Inventors: Greg L. Corpier, Sedalia, MO (US); Katie J. Boyer, Leavenworth, KS (US)

(21) Appl. No.: 15/276,639

(22) Filed: Sep. 26, 2016

Related U.S. Application Data
(63) Continuation of application No. 13/429,073, filed on Mar. 23, 2012, now abandoned.
(60) Provisional application No. 61/467,222, filed on Mar. 24, 2011.

(51) Int. Cl. G06F 19/00; G06F 17/30; A61B 5/00

(52) U.S. Cl. CPC G06F 19/325; A61B 5/0022; A61B 5/746; G06F 19/3406; G06F 19/3418; G06F 19/322; G06F 17/30312

(57) ABSTRACT

An automated healthcare integration system generally includes a plurality of residential stations, a clinician station, a data library, and may include emergency response stations. Each residential station includes a residential controller with analytical logic such as artificial intelligence, biometric devices interfaced to the residential controller, a user interface, and a communication interface for communication with the data library and the clinician station. The clinician station includes a clinician server, user interfaces, and a communication interface for communicating with the data library and the residential stations. Data derived from the biometric devices is communicated to and aggregated in the data library. Clinicians can retrieve such data for analysis to determine courses of treatment of the patients. Biometric and/or environmental sensor data indicating a life threatening situation of a patient can cause an alert to be issued to the emergency response station to assist such a patient.

### CROSS-REFERENCE TO RELATED APPLICATION

This application is a continuation of and claims priority in U.S. patent application Ser. No. 13/429,073, filed Mar. 23, 2012, which claims priority in U.S. Provisional Application No. 61/467,222, filed Mar. 24, 2011, both of which are incorporated herein by reference.

### BACKGROUND OF THE INVENTION

1. Field of the Invention

The present invention generally relates to healthcare information acquisition and management systems and, more particularly, to an integrated healthcare integration system which automates acquisition of healthcare information from a patient in the form of medical parameter measurements and queries, processes the information acquired, and responds to the processed information by conveying treatment and monitoring information to clinicians, enabling communication of the clinicians with the patients, conveying medical advice to the patient, acquiring further information from the patient, and issuing alerts to emergency services when appropriate.

2. Description of the Related Art

Persons with certain health conditions requiring close monitoring are often hospitalized or placed in other types of healthcare facilities. Such conditions may require frequent measurements of blood pressure, oxygen levels, sugar levels, and other medical data. These conditions may be chronic in nature or temporary, such as a result of a medical procedure, a disease, or the like. Placement in such facilities is usually a substantial financial burden to the patient, the patient's family, and the patient's insurer. Additionally, residence in such a facility separates the patient from his or her family members and friends, familiar surroundings, and preferred routines and activities.

Current medical technology provides a wide range of biometric devices for measuring and recording various parameters or vital signs of patients, such as body temperature, pulse rate, respiration rate, blood pressure, blood oxygen level, blood sugar level, cardiac waveforms, and many other factors. Many current biometric devices are digital in nature and can be interfaced to computers for periodically making such measurements, recording the measurements, comparing the measurements to established limits, and issuing warnings if the measurements are out of the established limits. The recorded measurements can be reviewed by clinicians, such as physicians, nurse practitioners, nurses, or the like, to monitor the current health of the patient and the progress or lack of progress of recuperation and to make changes to the course of treatment in response to trends which are discerned. Such systems of biometric devices are commonly used in hospitals, particularly in intensive care units, to enable nurses to monitor the conditions of many patients at a central station.

While such systems of biometric devices interfaced to computers have been in use in hospitals for a number of years, they are not readily available to patients in residential situations. Often, non-hospitalized patients must make measurements and manually record the parameters, such as blood pressure, prescriptions taken at specific times, or the like, on a health record. Some types of biometric devices store a record of measurements in a non-volatile memory. The chart or biometric device with memory is then taken to a clinician during an office visit where it is reviewed by the clinician. While such a manner of recording medical information is useful, it is often not sufficiently timely and may be subject to error if the patient incorrectly records a measurement or other data.

### SUMMARY OF THE INVENTION

The present invention provides embodiments of an automated healthcare integration system. The system generally includes a plurality of residential healthcare integration stations, a clinician station in communication with the residential stations, and a data library, in communication with the residential and clinician stations. In an embodiment of the system, each residential station includes a residential healthcare integration controller or computer, a plurality of biometric devices interfaced to the residential controller through wired and/or wireless biometric device interfaces, a residential user interface connected to the controller, and a residential remote communication interface connected to a large scale communication network, such as the public switched telephone network (PSTN) which provides data and voice communication services. The biometric devices are used to measure vital signs and other parameters of the patient, such as, but not limited to, body temperature, pulse rate, respiration rate, blood pressure, blood oxygen level, blood sugar level, cardiac waveforms, and other factors. The residential user interface may include devices such as a keyboard and computer display or touch based interface along with audio devices such as a microphone and a speaker.

In an embodiment of the system, the clinician station includes a clinician healthcare integration server having one or more clinician controllers or computers interfaced therewith. Each clinician controller includes a clinician user interface similar to the residential user interface. The clinician controller is connected by a clinician remote communication interface to the large scale communication network to enable communication with the residential stations.

The values of the patient parameters measured by the biometric devices are communicated from the residential stations to the clinician station where clinicians assigned to particular patients review the values and the history and trends of values. The clinician may adjust treatments, prescriptions, or the like for the particular patient, based on the combinations and trends of the patient parameters observed. Additionally, the clinician server can be programmed with analytical logic to process data received from the residential stations to enable the server to recognize symptoms, trends, and some diseases or pathologies based on the patterns of patient parameters. The analytical may also recognize improvements in various functions of the patients from the patterns of the parameters. The pathologies and clinical alerts recognized by the analytical logic may be suggested to the clinician for clinical significance or as a condition to be ruled out. The analytical logic may involve logic such as decision trees for various diseases or conditions and may also include artificial intelligence. Analysis of the patterns of patient parameters may suggest a need for additional biometric measurements, a different schedule for such measurements, changes in medications, changes in lifestyle activities, specific interventions, and the like. Advice or recommendations of the clinician can be conveyed to the patient over the communication as either text or as a real time online conference with the patient.

In addition to the measurements by the biometric devices, the residential controller may be provided with analytical logic to routinely query the patient regarding daily activities and overall health conditions of the patient. The analytical logic may include artificial intelligence programmed into the residential controller. Such queries may be text based or verbal, using speech recognition and speech synthesis incorporated into the residential user interface. Responses of the patient are recorded and conveyed to the clinician using the communication components of the healthcare integration system. A clinician can customize a branching question tree based on the condition or disease state of the patient.

In an embodiment of the automated healthcare integration system of the present invention, a data library is provided in communication with the residential stations and the clinician station and serves as a repository component of the system to store data received from the residential stations in association with the protected identities of the pertinent patients. The data from the residential stations, including measurements of physiological parameters and records of patient queries, are automatically communicated to the data library by the residential controllers. The patients normally do not have direct access to modify the data library. The clinicians are provided with access to the data associated with patients assigned to them for review and recommendations. The data library may be located at the clinician station or may be located in a secure location to provide for backup and redundancy and to serve multiple clinician stations.

Along with biometric and environmental measurements, patterns of patient parameters or replies to patient alerts may indicate immediate dangerous or life threatening situations. For this reason, the automated healthcare integration system of the present invention is provided with access to one or more emergency response services or stations, which may include fire, rescue, and ambulance services. In an embodiment of the system, the clinician station and the residential stations are provided with communication capabilities to the emergency response stations. Preferably, the analytical logic programming of the residential controllers and artificial intelligence are provided with logic for assessing such dangerous or life threatening conditions. Additionally, the analytical logic programming of the residential controller and artificial intelligence may be provided with routines for determining the need for a visiting clinician to a patient or to suggest the need for an office visit and live consultation with a clinician.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a block diagram illustrating the principal components of an automated healthcare integration system according to the present invention.

FIG. 2 is a block diagram illustrating principal components of an embodiment of a clinician station according to the present invention.

FIG. 3 is a block diagram illustrating principal components of a residential station according to the present invention.

FIG. 4 is a block diagram illustrating software components of an embodiment of a residential healthcare integration controller of the present invention.

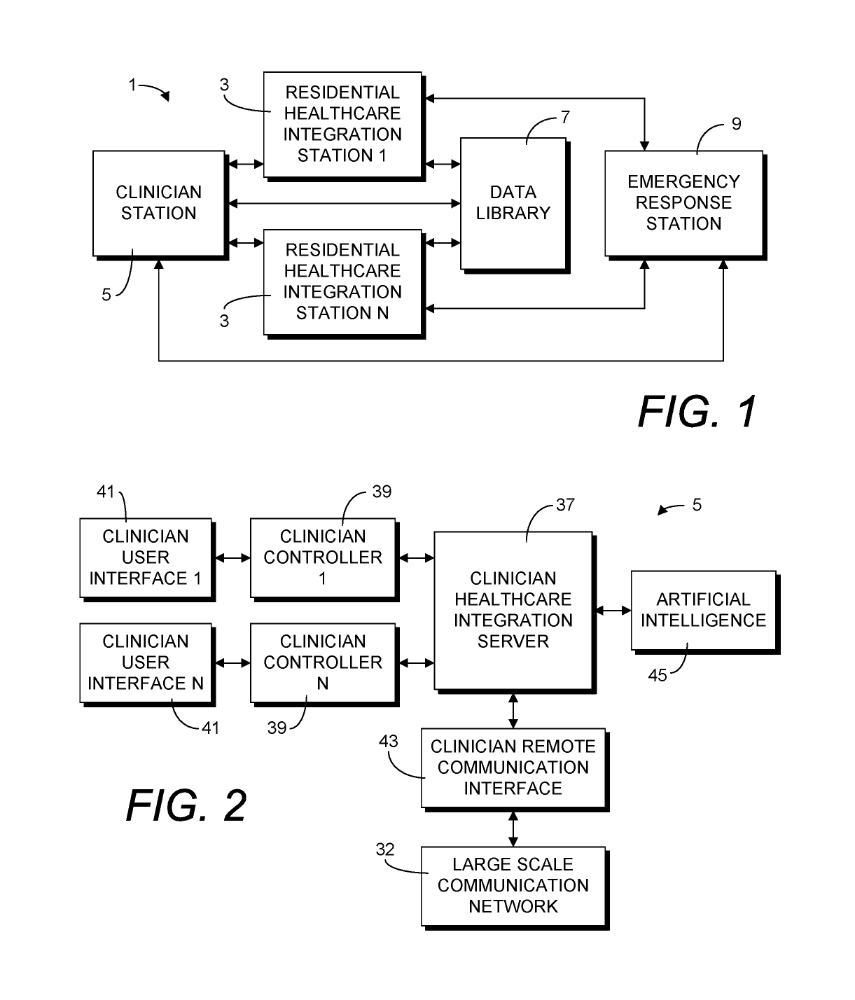

### DETAILED DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring now to the drawings in more detail, the reference number 1 generally designates an embodiment of an automated healthcare integration system according to the present invention. The system 1 generally includes a plurality of residential healthcare integration stations 3 which communicate with a clinician station 5 and a healthcare integration data library 7. The residential stations 3 are located at the residences of patients or locations commonly occupied by the patients, while the clinician station 5 and data library 7 are remote from the residential stations 3. Generally, the residential stations 3 enable the collection of patient parameters such as physiological and other data from the patients, automatic communication of such data from the residential stations 3 to the clinician station 5 or data library 7 to enable clinicians at the clinician station 5 to assess the condition of the patients and make recommendations about schedules of parameter measurements, lifestyle activities, clinician office visits, and the like. The system 1 may include an emergency response station 9 communicating with the clinician station 5 and the residential stations 3. The emergency response station 9 can be alerted by the clinician station 5 or a residential station 3 in response to a clinician determining the necessity of emergency action based on a review of patient parameters or upon an acute event occurring to a patient at a residence.

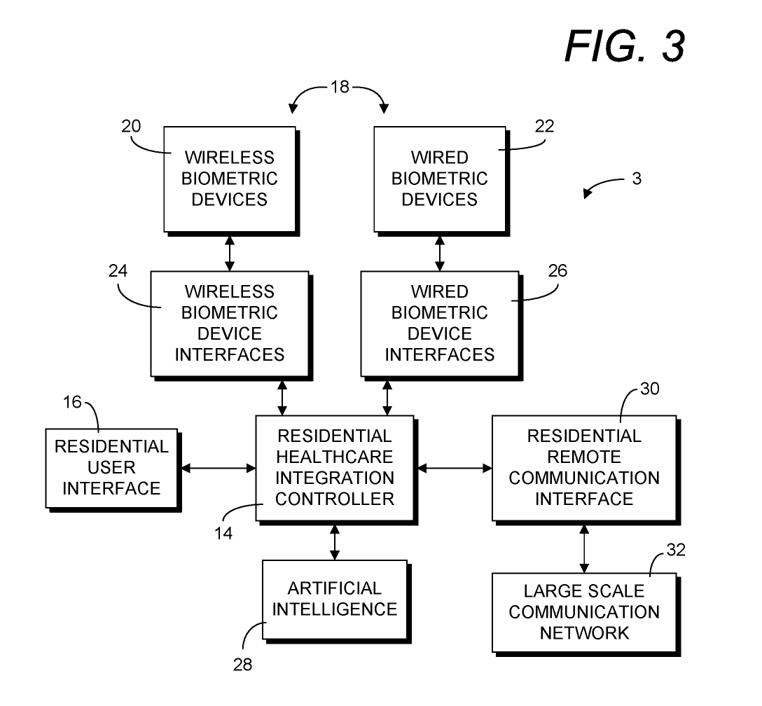

Referring to FIG. 3, an embodiment of a residential healthcare integration station 3 includes a residential healthcare integration controller or computer 14 having a residential user interface 16 connected thereto. The controller 14 and user interface 16 can be implemented as a personal computer, such as desktop computer with a keyboard, display, microphone, and speaker (not shown), a laptop computer, an “all-in-one” computer, a tablet computer, a smart phone, or the like. The controller 14 includes a central processing unit (not shown) and data storage components (not shown) storing an operating system, programs, and data. Personal computers are well known in the data processing arts and should be familiar to those skilled in healthcare data processing.

The illustrated residential station 3 includes biometric devices 18 which are interfaced to the residential controller 14. The biometric devices 18 may include wireless biometric devices 20 and/or wired biometric devices 22. The biometric devices 18 are devices for measuring vital signs and parameters of the patient at the associated residence. The biometric devices 18 include instruments for measuring such parameters as, but not limited to, body temperature, body weight, pulse rate, respiration rate, blood pressure, blood oxygen level, blood sugar level, breath analysis, cardiac waveforms, brain waves, and other factors. The wireless biometric devices 20 communicate with the residential controller 14 by wireless biometric device interfaces 24. The wireless interfaces 24 may include wireless interface technologies, such as Wi-Fi™ (Wi-Fi Alliance, www.wi-fi.org), Bluetooth™ (Bluetooth Special Interest Group, www.bluetooth.com), ZigBee™ (ZigBee Alliance, www.zigbee.org), infrared links, and other types of wireless technologies. Similarly, the wired biometric devices 20 communicate with the residential controller 14 by means of wired biometric device interfaces 26. The wired interfaces 26 may include wired interface technologies, such as universal serial bus or USB, Ethernet, serial interfaces such as RS-232 or RS-485, and other wired interface technologies.

The residential controller 14 is programmed with analytical logic to store data from the biometric devices 18 and to analyze the data for trends. The analytical logic may include such programming as artificial intelligence 28, decision trees, or the like for identifying symptoms of some syndromes, pathologies, diseases, and the like. In addition to recording and analyzing data from the biometric devices 18, the residential controller 14 is capable of making selected queries of the patient and recording responses, by way of the residential user interface 16, and recording responses to the queries. The queries may be made in the form of text or speech queries and responses. In order to enable speech queries and responses, the residential controller 14 is provided with speech recognition and speech synthesis capabilities. The analytical logic may incorporate analysis of responses to such queries in the analysis of data from the biometric devices 18.

Artificial intelligence is a system of programs and data structures that simulate human reaction and performance of tasks in a particular environment. This simulation includes the ability to learn via sensory inputs and multiple methods of feedback. The current embodiment of the residential station 3 may utilize several algorithms including finite state modeling, virtual environment modeling, rules based inference and an expert system, genetic algorithms, and weighted responses based on feedback. Through the creation of a virtual simulation of the patient data built from biometric device data, historical data, and patient queries/responses, the simulation can achieve “situational awareness” and make decisions and calculations based on all the data available. The simulation can also run “what if” scenarios in virtual space to determine what action is the “best to use” in the situation at hand. Each of the scenarios may be applied to the genetic algorithms to determine the best result and each may be applied to the weighted responses to allow the simulation to “learn”. Additional information disclosing aspects and uses of artificial intelligence can be found in U.S. Pat. Nos. 5,673,637; 7,263,509; and 7,389,208, which are incorporated herein by reference.

In order to communicate data from the biometric devices 18 and query data to the data library 7, the residential controller is provided with a residential remote communication interface 30 which provides communication over a large scale communication network 32 such as the public switched telephone network (PSTN) which provides data and voice service. It should be noted that the data library 7 incorporates a data library remote communication interface (not shown) to the large scale communication network 32. The residential communication interface 30 may be capable of wired broadband service, wireless broadband service, and/or dial-up service. The wired broadband service may include digital subscriber line (DSL), very-high-bit rate digital subscriber line (VDSL), cable modem, fiber optic service, or the like. The wireless broadband service may include various kinds of cellular data communication protocols such as code division multiple access (CDMA), global system for mobile communications (GSM), or third or fourth generation cellular data communication protocols (3G or 4G), or the like. Dial-up capability can be provided in the residential communication interface 30 for backup communication when the broadband services are not available. It is foreseen that the residential controller 14 may communicate with the residential communication interface 30 wirelessly.

Referring to FIG. 2, an embodiment of a clinician station 5 includes a clinician healthcare integration server 37 having a plurality of clinician controller or computers 39 interfaced thereto. Each clinician controller 39 has a clinician user interface 41 communicating therewith. Each combination of a clinician controller 39 and associated clinician interface 41 can be implemented as a personal computer, such as desktop computer, a laptop computer, an “all-in-one” computer, or the like, each with a keyboard, display, microphone, and speaker (not shown). The clinician server 37 communicates with the large scale communication network 32 by way of a clinician remote communication interface 43, which may employ the same communication protocols as the residential remote communication interface 30. For reliability and high data throughput, wired broadband services are preferred in the illustrated clinician remote communication interface 43. While the clinician station 5 is normally located situated in a building, such as a medical office building, it is foreseen that mobile clinician stations 5 can also be implemented. In such a mobile situation, the clinician controller 39 would be provided with communications to the clinician server 37 using one or more of the mobile communication technologies described above.

Clinicians at the clinician station 5 periodically retrieve patient data from the data library 7 regarding patients assigned to them. The clinicians may review the raw data from the biometric devices 18 at the residential stations 3 or may apply such data to analytical programming to detect patterns, trends, or the like which may indicate good health of the patient, changes in certain patient functions such as improvements or deteriorations. Additionally, the clinicians need to determine if previously unknown problems are occurring with their patients. For these purposes, an embodiment of the clinician server 37 is provided with clinician artificial intelligence programming 45 which may incorporate analytical processing similar to the residential artificial intelligence programming 28, in addition to other capabilities.

Based on the clinician's diagnosis of the patient's condition from the raw biometric data and queries, the clinician can make recommendations ranging from maintaining the current regimens, making more frequent or different biometric measurements and/or patient queries, recommending a visit to the patient's residence by a clinician, or recommending a visit to a clinician's office or the clinician station 5. The diagnosis may also generate a voice call to the patient from the clinician for queries and responses. Under certain circumstances, analysis of the biometric data by the server 37 and/or a clinician may indicate a dangerous or life-threatening situation of a patient, thereby triggering an alert to the emergency response station 9.

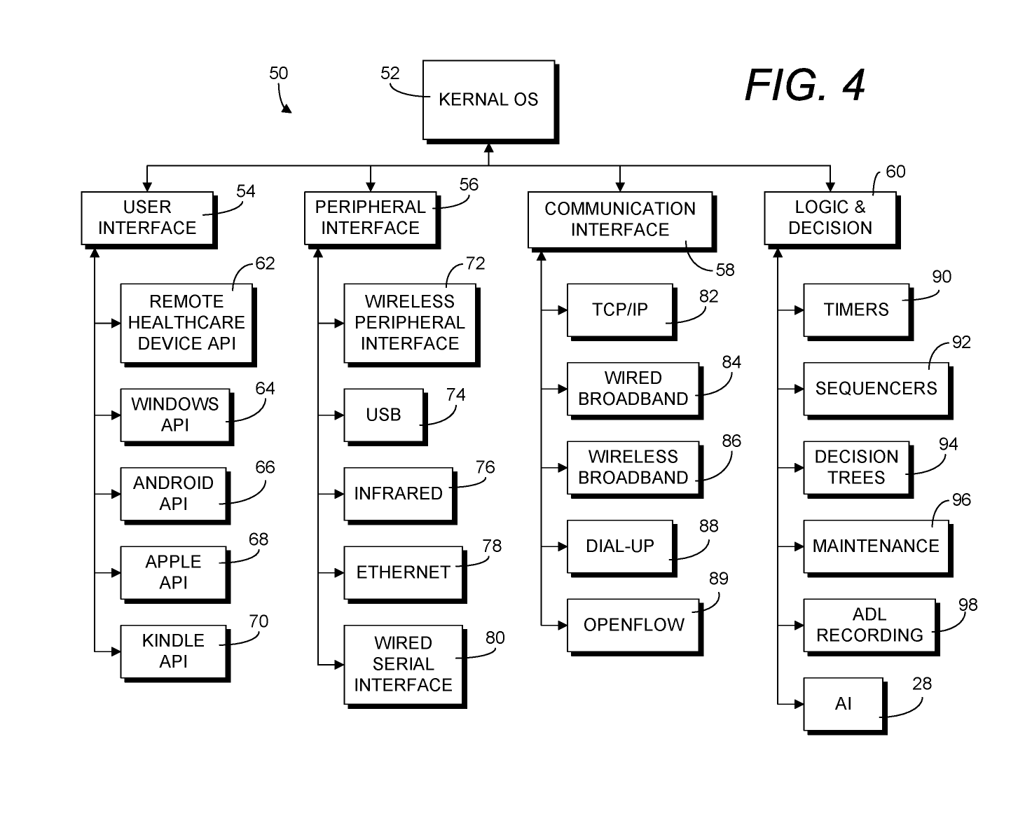

FIG. 4 illustrates an embodiment of software components 50 which may be executed or accessed by the residential controller 14. FIG. 4 illustrates many components, not all of which may be present in every residential controller 14. The software components 50 include a kernel operating system or OS 52 functions to run the core system operations of the residential controller 14. These operations are divided into four logical and virtual layers: a user interface layer 54, a peripheral interface layer 56, a communication interface layer 58, and a logic and decision layer 60.

The user interface layer 54 is a high-layer virtual to physical communication layer between the patient and the residential controller 14. It provides access to the routines that record data from the biometric devices 18 and queries and responses by use of the physical user interface 16. The user interface layer 54 is formed by one or more application programming interfaces or API's and may include a remote healthcare device API 62, similar to that described in U.S. patent application Ser. No. 13/306,755 for AUTOMATED PERSONAL ASSISTANCE SYSTEM, filed Nov. 29, 2011, which is incorporated herein by reference, a Windows™ API 64 (Microsoft, Inc. www.microsoft.com), an Android™ API 66 (Google, Inc. www.google.com), an Apple™ API 68 (Apple Computer, Inc. www.apple.com), a Kindle™ API 70 (Amazon Technologies, Inc. www.amazon.com), and/or the like.

The peripheral interface layer 56 provides virtual messaging for communication of the biometric devices 18 with the residential controller 14. The peripheral interface layer 56 may include one or more of the following peripheral interface components: wireless peripheral interfaces 72, such as Wi-Fi™, Bluetooth™, ZigBee™, or the like; universal serial bus 74 (USB), infrared 76, Ethernet 78, wired serial interfaces 80 such as RS-232 or RS-485, or the like.

The communication interface layer 58 facilitates the communication of data from the residential controller 14 to the data library 7 or the clinician station 5 and may include components such as transmission control protocol and internet protocol (TCP/IP) 82, commonly referred to as simply internet protocol; wired broadband 84 such as digital subscriber line (DSL), very-high-bit rate digital subscriber line (VDSL), cable modem, fiber optic service, or the like; wireless broadband 86 such as code division multiple access (CDMA), global system for mobile communications (GSM), or third or fourth generation cellular data communication protocols (3G or 4G), or the like; dial-up 88, and/or OpenFlow™ protocol 89 (Open Networking Foundation www.opennetworking.org).

The logic and decision layer 60 provides certain types of intelligence to the residential controller 14 and may include timers 90 that start device timeouts when the patient is instructed to perform a task. A timeout is required to make sure that the task is performed in a suitable or critical amount of time or performed at all. The layer 60 may include sequencers 92 which control information flow by using biometric device settings to set the timers 90 when performing a sequence of instructions and staging when to run sets of instructions. The layer 60 may include decision trees 94 which are sets of questions based on the disease for which the patient is being treated. The questions can relate, for example, to chronic obstructive pulmonary disease (COPD), diabetes, or the like. There may be multiple decision trees 94 which contain different sets of rules and logic. The layer 60 may include maintenance components 96 which control the manner in which information is updated. For example, the maintenance function 96 can determine how clinicians add new decision trees and when additional biometric devices 18 need to be added to a residential station 3. The layer 60 may include ADL recording 98, that is, the recording of information related to activities of daily living. This function may capture motions of the patient, based on tracking sensors (not shown) and tracks behavior patterns in self-care activities within a patient's residence, in a facility, or anywhere tracking is available. Activities of daily living may include things the patient normally engages in such as eating, bathing, dressing, grooming, sleeping and the like. Such activities may also include tasks such as balancing a checkbook, making a grocery list, leisure activities, and the like. Finally, the logic and decision layer 60 may include the artificial intelligence programming 28, as described above.

While the foregoing written description of embodiments of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. And while certain forms of the present invention have been illustrated and described herein, it is not to be limited to the specific forms or arrangement of parts described and shown.

Having thus described the invention, what is claimed and desired to be secured by Letters Patent is:

1. An automated healthcare integration system comprising:
(a) a residential healthcare integration station positioned at a residence of a patient and including:
(1) a residential healthcare integration controller;
(2) a biometric device interfaced to said residential controller and being adapted for measuring a patient parameter of said patient;
(3) a residential user interface communicating with said residential controller; and
(4) a residential remote communication interface communicating with said residential controller to communicate measurements of said parameter;
(b) a clinician healthcare integration station positioned remotely from said residential station and including:
(1) a clinician healthcare integration server;
(2) a clinician user interface communicating with said clinician server for use by a clinician at said clinician station; and
(3) a clinician remote communication interface communicating with said clinician server and with said residential communication interface; and
(c) said residential station cooperating with said clinician station to enable communication of measurements of said patient parameter of said patient to said clinician, review of said patient parameter and a history thereof over a period of time by said clinician, and communication of medical directions from said clinician to said patient regarding said history of said patient parameter.

2. A system as set forth in claim 1 and including:
(a) a data library in communication with said residential remote communication interface and said residential remote communication interface and storing said history of said patient parameter in association with the identity of said patient.

3. A system as set forth in claim 1 and including:
(a) a plurality of residential healthcare integration stations in communication with said clinician station.

4. A system as set forth in claim 1 and including:
(a) a plurality of residential healthcare integration stations in communication with said clinician station; and
(b) a plurality of clinician user interfaces communicating with said clinician controller to enable a plurality of clinicians to monitor and respond to patient parameters of a plurality of respective patients associated with said plurality of residential stations.

5. A system as set forth in claim 1 and wherein said residential healthcare integration station includes:
(a) a wired biometric device; and
(b) a wired biometric device interface communicating with said residential healthcare integration controller to communicate a measurement of said patient parameter thereto.

6. A system as set forth in claim 1 and wherein said residential healthcare integration station includes:
(a) a wireless biometric device; and
(b) a wireless biometric device interface communicating with said residential healthcare integration controller to communicate a measurement of said patient parameter thereto.

7. A system as set forth in claim 1 and including:
(a) an emergency response station interfaced to said residential station and said clinician station to enable dispatching emergency personnel to said residential station in response to an emergency condition of said patient as determined by said patient or said clinician.

8. A system as set forth in claim 1 and including:
(a) said clinician user interface cooperating with said residential user interface through said clinician controller and said residential controller to enable said clinician to selectively query said patient regarding conditions of said patient and said patient to respond to said query from said clinician.

9. A system as set forth in claim 1 and including:
(a) said residential controller being programmed with artificial intelligence to enable said residential controller to learn patterns of said patient parameter over time and to report said patterns to said clinician station.

10. A system as set forth in claim 1 and including:
(a) said clinician server being programmed with artificial intelligence to enable said server to learn patterns of patient parameters associated with patients over time and to provide access to said patterns by a clinician.

11. An automated healthcare integration system comprising:
(a) a plurality of residential healthcare integration stations positioned respectively at residences of associated patients, each residential station including:
(1) a residential healthcare controller;
(2) a biometric device interfaced to said residential controller and being adapted for measuring a patient parameter of an associated patient;
(3) a residential user interface communicating with said residential controller; and
(4) a residential remote communication interface communicating with said residential controller to communicate measurements of said parameter;
(b) a clinician healthcare integration station positioned remotely from said residential station and including:
(1) a clinician healthcare integration controller;
(2) a clinician user interface communicating with said clinician controller for use by a clinician at said clinician station; and
(3) a clinician remote communication interface communicating with said clinician controller and with said residential communication interface; and
(c) said residential stations cooperating with said clinician station to enable communication of measurements of said patient parameters of said associated patient to said clinician, review of said patient parameters and histories thereof over periods of time by said clinician, and communication of medical directions from said clinician to said patient regarding said history of physical parameters associated therewith.

12. A system as set forth in claim 11 and including:
(a) a data library in communication with said residential remote communication interface and said residential remote communication interface and storing said histories of said patient parameters in association with the identities of said patients.

13. A system as set forth in claim 11 and including:
(a) a plurality of clinician user interfaces communicating with said clinician controller to enable a plurality of clinicians to monitor and respond to patient parameters of a plurality of respective patients associated with said plurality of residential stations.

14. A system as set forth in claim 11 and wherein at least one of said residential healthcare integration stations includes:
(a) a wired biometric device; and
(b) a wired biometric device interface communicating with said residential healthcare integration controller to communicate a measurement of said patient parameter thereto.

15. A system as set forth in claim 11 and wherein at least one of said residential healthcare integration stations includes:
(a) a wireless biometric device; and
(b) a wireless biometric device interface communicating with said residential healthcare integration controller to communicate a measurement of said patient parameter thereto.

16. A system as set forth in claim 11 and including:
(a) an emergency response station interfaced to said residential station and said clinician station to enable dispatching emergency personnel to one of said residential station in response to an emergency condition of the patient associated with said one of said residential station as determined by the associated patient or said clinician.

17. A system as set forth in claim 11 and including:
(a) said clinician user interface cooperating with said residential user interfaces through said clinician controller and said residential controllers to enable said clinician to selectively query said patients regarding conditions of said patients and said patients to respond to said query from said clinician.

18. A system as set forth in claim 11 and including:
(a) each of said residential controllers being programmed with artificial intelligence to enable said residential controller to learn patterns of said patient parameter over time and to report said patterns to said clinician station.

19. A system as set forth in claim 11 and including:
(a) said clinician server being programmed with artificial intelligence to enable said server to learn patterns of patient parameters associated with said patients over time and to provide access to said patterns by a clinician.

20. An automated healthcare integration system comprising:
(a) a plurality of residential healthcare integration stations positioned respectively at residences of associated patients, each residential station including:
(1) a residential healthcare controller;
(2) a biometric device interfaced to said residential controller and being adapted for measuring a patient parameter of an associated patient;
(3) a residential user interface communicating with said residential controller; and
(4) a residential remote communication interface communicating with said residential controller to communicate measurements of said parameter;
(b) a clinician healthcare integration station positioned remotely from said residential station and including:
(1) a clinician healthcare integration controller;
(2) a clinician user interface communicating with said clinician controller for use by a clinician at said clinician station; and
(3) a clinician remote communication interface communicating with said clinician controller and with said residential communication interface;
(c) said residential stations cooperating with said clinician station to enable communication of measurements of said patient parameters of said associated patient to said clinician, review of said patient parameters and histories thereof over periods of time by said clinician, and communication of medical directions from said clinician to said patient regarding said history of patient parameters associated therewith;
(d) a data library in communication with said residential remote communication interface and said residential remote communication interface and storing said histories of said patient parameters in association with the identities of said patients;
(e) an emergency response station interfaced to said residential station and said clinician station to enable dispatching emergency personnel to one of said residential station in response to an emergency condition of the patient associated with said one of said residential station as determined by the associated patient or said clinician;
(f) a plurality of clinician user interfaces communicating with said clinician controller to enable a plurality of clinicians to monitor and respond to patient parameters of a plurality of respective patients associated with said plurality of residential stations;
(g) a wireless biometric device;
(h) a wireless biometric device interface communicating with said residential healthcare integration controller to communicate a measurement of said patient parameter thereto;
(i) said clinician user interface cooperating with said residential user interfaces through said clinician controller and said residential controllers to enable said clinician to selectively query said patients regarding conditions of said patients and said patients to respond to said query from said clinician;
(j) each of said residential controllers being programmed with artificial intelligence to enable said residential controller to learn patterns of said patient parameter over time and to report said patterns to said clinician station; and
(k) said clinician server being programmed with artificial intelligence to enable said server to learn patterns of patient parameters associated with said patients over time and to provide access to said patterns by a clinician.
